(19) United States
(12) Patent Application Publication
(10) Pub. No.: US 2010/0292671 A1
(43) Pub. Date: Nov. 18, 2010

(54) In Vivo Payload Delivery Apparatus and Method

(75) Inventors: Fred A. Allgood, Fort Worth, TX (US); Ross Teggatz, McKinney, TX (US); Wayne T. Chen, Plano, TX (US); Nick Papacharalambous, Austin, TX (US)

Correspondence Address: MICHAEL T. KONCZAL, PATENT ATTORNEY, P.O. BOX 863656, PLANO, TX, 75086, US

(73) Assignees: TRIUNE IP LLC, Richardson, TX (US); TAP PRODUCTS LLC, Richardson, TX (US)

(21) Appl. No.: 12/782,529

(22) Filed: May 18, 2010

Related U.S. Application Data
(60) Provisional application No. 61/179,175, filed on May 18, 2009.

(51) Int. Cl. A61M 31/00

(52) U.S. Cl. 604/515; 604/60

(57) ABSTRACT

The disclosed invention provides apparatus and methods for delivering a payload to a target location inside the body. A finger board is retained on the finger of a user, having a proximal end and a distal end. A payload holder is included at the distal end of the finger board for holding a payload during positioning and prior to delivery. An ejector is included for the purpose of ejecting the payload from the payload holder when actuated by a user.

### PRIORITY ENTITLEMENT

This application is entitled to priority based on Provisional Patent Application Ser. No. 61/179,175 filed on May 18, 2009, which is incorporated herein for all purposes by this reference. This application and the Provisional Patent Application have at least one common inventor.

### TECHNICAL FIELD

The invention relates to apparatus and methods for delivering payloads in hard-to-access locations. More particularly, the invention relates to apparatus and methods for depositing medication or other payload at a selected location in vivo, that is, inside the human body.

### BACKGROUND OF THE INVENTION

In some contexts, there are occasions in which it is desirable to place objects in hard-to-access locations. Oftentimes, such efforts are hampered by challenges in manipulating the objects and delivering them to locations that are out of the line-of-sight. For example, in the medical field, there are instances where it is desirable to apply medication in a specific site in vivo, in locations where access through a natural body orifice is often difficult. It is known in the art to apply such medication by hand, using physical touch to identify the correct location. Practitioners are capable of depositing medication in precise locations depending in large part on the depth of their experience and other factors such as the time and facilities available, as well as the specific physiology and condition of the patient. An example of the deposition of medication within a body cavity is the administration of Cervidil (a registered trademark of Forest Laboratories Inc., N.Y.). This medication is used during childbirth for loosening up the female cervix to facilitate delivery. The medication packet is designed to be deposited in close proximity to the cervix. A tape attached to the medication packet is used for later withdrawal. The usual method of application is for a medical professional to wedge the medicine between the index and middle fingers for insertion into the vagina. Following insertion, the medical professional then feels for the correct position in close proximity to the cervix, releases the medication from between the fingers at the target location, and then withdraws the fingers. This commonly used approach poses several problems. It is usually difficult to deposit the medication in precisely the desired location. Considerable manual dexterity is required for holding the medication somewhat awkwardly between the fingers, feeling for the correct location based on knowledge, training, and experience, and then depositing the medicine and carefully withdrawing the fingers while avoiding mis-positioning the medication or inadvertently pulling it out by the attached tape. The difficulties with this procedure sometimes lead to incorrect positioning of the medication, unintended withdrawal, patient discomfort, or dropped or damaged medication packets, leading in turn to increased costs.

Due to the foregoing and possibly additional problems, improved apparatus and methods for depositing medication or other objects within body cavities would be a useful contribution to the arts.

### SUMMARY OF THE INVENTION

In carrying out the principles of the present invention, in accordance with preferred embodiments, the invention provides advances in the arts with useful and novel apparatus and methods for depositing payloads within hard-to-access locations. Embodiments described herein include apparatus and methods particularly well-adapted for administering Cervidil as examples of preferred implementations of the invention. The embodiments described herein are intended to be exemplary and not exclusive. Variations in the practice of the invention are possible and preferred embodiments are illustrated and described for the purposes of clarifying the invention. All possible variations within the scope of the invention cannot, and need not, be shown.

According to one aspect of the invention, apparatus for delivering a payload in a hard-to-access location has a finger board with a retainer for securing the apparatus to a finger of a user. A payload holder is included at the distal end of the finger board for holding a payload during positioning prior to delivery. An ejector is secured to the finger board and is operable from outside of the body for ejecting the payload at the target site within.

According to another aspect of the invention, apparatus for delivering a payload in vivo is particularly adapted for depositing medication in close proximity to the cervix. The apparatus includes a finger board, retainer for securing the apparatus to a finger of a user, and a payload holder adapted for holding a medication payload during positioning adjacent to the cervix. An ejector secured to the finger board is operable for ejecting the payload.

According to yet another aspect of the invention, in further variations of the preferred embodiments, methods for delivering payloads in the proximity of the cervix include steps for deploying apparatus further described herein.

The invention has advantages including but not limited to providing one or more of the following features, reduced patient discomfort, reduced medical personnel training and/or experience requirements, improved success in depositing payloads in the correct locations, reduced waste due to misplacement and/or inadvertent withdrawal, reduced costs. These and other advantages, features, and benefits of the invention can be understood by one of ordinary skill in the arts upon careful consideration of the detailed description of representative embodiments of the invention in connection with the accompanying drawings.

### BRIEF DESCRIPTION OF THE DRAWINGS

The present invention will be more clearly understood from consideration of the description and drawings in which:

FIG. 1 is a perspective view of an example of a preferred embodiment of apparatus according to the invention; and

FIG. 2 is a perspective view of another example of apparatus according to a preferred embodiment of the invention.

References in the detailed description correspond to like references in the various drawings unless otherwise noted. Descriptive and directional terms used in the written description such as front, back, top, bottom, upper, side, et cetera; refer to the drawings themselves as laid out on the paper and not to physical limitations of the invention unless specifically noted. The drawings are not to scale, and some features of embodiments shown and discussed are simplified or amplified for illustrating principles and features as well as advantages of the apparatus and related methods of the invention.

### DESCRIPTION OF PREFERRED EMBODIMENTS

While the making and using of various exemplary embodiments of the invention are discussed herein, it should be appreciated that the apparatus and techniques for its use exemplify inventive concepts which can be embodied in a wide variety of specific contexts. It should be understood that the invention may be practiced in various applications and embodiments without altering the principles of the invention. For purposes of clarity, detailed descriptions of functions, components, and systems familiar to those skilled in the applicable arts are not included. In general, the invention provides apparatus and methods for depositing payloads at selected target locations in body cavities. The invention is described in the context of representative example embodiments. Although variations and alternatives for the details of the embodiments are possible, each has one or more advantages over the prior art.

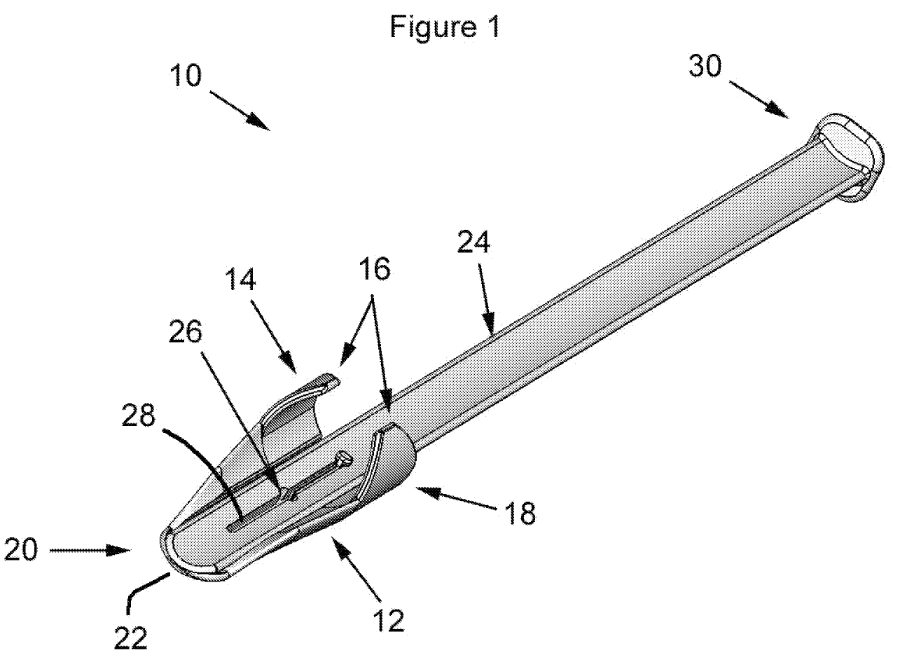

An example of a preferred embodiment of apparatus for depositing medication in vivo is portrayed in FIG. 1. The apparatus 10 includes a finger board 12 configured for securing to the finger of a user (not shown). A retainer arrangement 14, such as the tabs 16 shown in this example, secures the apparatus 10 to the finger. At its proximal end 18, the finger board 12 preferably terminates short of the user's palm. At its distal end 20, the finger board 12 preferably extends beyond the user's finger to some extent. As shown at reference numeral 22, a payload holder is provided for retaining medication prior to its dispensation at the appropriate location. The payload holder 22 may comprise an area at the distal end 20 of the finger board 12. Variations of the configuration of the payload holder 22 may be made without departure from the scope of the invention. The selection of a suitable payload holder 22 configuration may be made for a particular implementation of the apparatus based upon the location and nature of the medication, or other payload, intended for deposition. The example of FIG. 1 illustrates a payload holder 22 suitable for use with a solid object such as a pill, tablet, or capsule, which may be retained between the user's finger and the payload holder 22. An ejector 24 is preferably operable to assist with the ejection of the medication from the payload holder 22, in this embodiment by sliding longitudinally, namely retracting from the distal end 20 of the finger board 12, such that any payload present on the payload holder 22 would be ejected as the ejector 24 is withdrawn. As shown in the exemplary embodiment of FIG. 1, the ejector 24 may be connected with the finger board using a pin 26 and slot 28 arrangement whereby the ejector 24 is affixed to the fingerboard 12 but is free to slide to the extent allowed by the slot 28.

In an example of the use of the invention, a user preferably secures the apparatus 10 by inserting a finger into the retainer 14 and placing the finger board 12 on the finger. A dosage of medication is placed in the payload holder 22. The user then maneuvers the apparatus into the desired position inside the hard-to-access location, in this case in vivo, preferably using the finger as a guide and as a reference. Upon reaching the target, the user activates the ejector 24, preferably by pushing or pulling its proximal end 30 with the opposite hand, depositing the medication at the targeted location. Those skilled in the arts should appreciate that the apparatus facilitates the use of a single finger to hold and position the payload, which takes up less room and is more easily maneuvered within body cavities, providing potential advantages in terms of ease of use, precision, speed, and reduced patient discomfort. The small cross-section provides additional advantages in some applications, particularly those in which a withdrawal tape or string is used. For example, when used to deposit Cervidil in close proximity to the cervix, the apparatus and associated finger may preferably be withdrawn off to one side of the canal, thus avoiding pulling on the withdrawal tape and thereby reducing the likelihood of the Cervidil becoming dislodged.

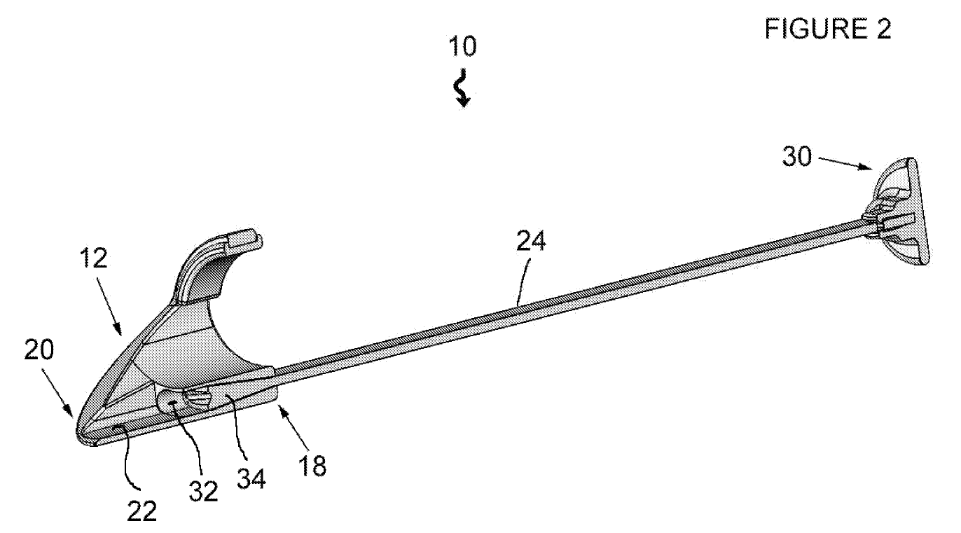

Examples of some of the alternative embodiments of apparatus within the scope of the invention may include various configurations of payload holders designed for different payload types. Referring primarily to FIG. 2, an alternative embodiment of apparatus 10 according to the invention is illustrated in a cutaway perspective side view. As shown in this configuration, the ejector shaft 24 may be captured by a channel 32 in the finger board 12. The payload holder 22 is preferably positioned such that the user's finger may be placed on top of a payload carried thereon prior to dispensation. It can also be seen that the ejector 24 may include a distal end 34 shaped such that it cannot pass through the channel 32 out of the proximal end 18 of the finger board 12. Also shown in this alternative embodiment, the retainer arrangement 14 may be annulus-shaped, retaining the apparatus 10 on the finger of the user similar to the wearing of a ring.

The apparatus and methods of the invention provide one or more advantages including but not limited to one or more of; increased accuracy and success in the placement of payloads in vivo, decreased training time for technicians or medical personnel, providing a cost-effective, preferably disposable device, facilitating faster treatment or shorter down times, and reduced costs. While the invention has been described with reference to certain illustrative embodiments, those described herein are not intended to be construed in a limiting sense. For example, variations or combinations of features or materials in the embodiments shown and described may be used in particular cases without departure from the invention. Although the presently preferred embodiments are described herein in terms of particular examples, modifications and combinations of the illustrative embodiments as well as other advantages and embodiments of the invention will be apparent to persons skilled in the arts upon reference to the drawings, description, and claims.

We claim:

1. Apparatus for delivering a payload in vivo comprising:
a finger board having one or more retainer adapted for securing the apparatus to a finger of a user, the finger board also having a proximal end and a distal end;
a payload holder at the distal end of the finger board for holding a payload during positioning prior to delivery; and
an ejector secured to the finger board and operable for ejecting the payload from the payload holder, the ejector configured for actuation at the proximal end.

2. The apparatus of claim 1 wherein the ejector comprises a plunger slidably secured longitudinally to the finger board.

3. The apparatus of claim 1 further comprising a longitudinal channel on the finger board for receiving a slidable ejector.

4. The apparatus of claim 1 wherein the payload holder further comprises a payload platform.

5. The apparatus of claim 1 substantially comprising an inexpensive disposable material.

6. The apparatus of claim 1 substantially comprising injection molded plastic.

7. Apparatus for placing medication in close proximity to the cervix comprising:
a finger board having one or more retainer adapted for securing the apparatus to a finger of a user, the finger board also having a proximal end and a distal end;
a payload holder at the distal end of the finger board for holding medication during positioning prior to placement; and
an ejector secured to the finger board for ejecting medication from the payload holder, the ejector configured for actuation at the proximal end.

8. The apparatus of claim 7 wherein the ejector comprises a plunger slidably secured longitudinally to the finger board.

9. The apparatus of claim 7 further comprising a longitudinal channel on the finger board for receiving a slidable ejector.

10. The apparatus of claim 7 wherein the payload holder further comprises a payload platform.

11. The apparatus of claim 7 substantially comprising an inexpensive disposable material.

12. The apparatus of claim 7 substantially comprising injection molded plastic.

13. A method for positioning and depositing a payload in vivo comprising the steps of:
wearing an apparatus having a finger board, the finger board having one or more retainer adapted for securing the apparatus to a finger of a user, the finger board also having a proximal end and a distal end;
placing a payload on a payload holder at the distal end of the finger board; thereafter,
positioning the apparatus at a target location in vivo; and
ejecting the payload from the payload holder; and thereafter
removing the apparatus from in vivo.

14. The method according to claim 13 further comprising the step of holding the payload on the payload holder during positioning.

15. The method according to claim 13 wherein the step of ejecting the payload from the payload holder further comprises actuating an ejector of the apparatus from outside the body.

16. The method according to claim 13 further comprising the step of disposing of the apparatus subsequent to a single use.

17. The method according to claim 13 further comprising the step of selecting a cervix as a target location.

18. The method according to claim 13 further comprising the step of selecting a gynecological medicament as the payload.

19. The method according to claim 13 further comprising the step of selecting an obstetric medicament as the payload.

20. The method according to claim 13 further comprising the step of selecting Cervidil as the payload.
